SEQUENCE LISTING (2 sequences)

SEQ ID NO: 1 - protein, 403 aa, Homo sapiens [sequence omitted]

SEQ ID NO: 2 - DNA, 1209 bp, Homo sapiens
atgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaacgaacctgatcattatagatattctgacaccactgactctgatccagagaatgaaccttttgatgaagatcagcatacacaaattacaaaagtc

(19) United States
(12) Patent Application Publication
(10) Pub. No.: US 2008/0305181 A1
(43) Pub. Date: Dec. 11, 2008

(54) Compositions and Methods for Treatment of Vascular Grafts

(76) Inventors: Christopher D. Kontos, Durham, NC (US); Jianhua Huang, Durham, NC (US)

Correspondence Address: Peter G. Carroll;Medlen & Carroll, 101 Howard Street, Suite 350, San Francisco, CA, 94105, US

(21) Appl. No.: 11/578,490

(22) PCT Filed: Apr. 18, 2005
(86) PCT No.: PCT/US05/13140

§ 371 Date: Feb. 4, 2008

Related U.S. Application Data
(60) Provisional application No. 60/563,726, filed on Apr. 20, 2004.

(51) Int. Cl. A61K 35/34; A61F 2/06; A61K 31/7088

(52) U.S. Cl. 424/569; 600/36; 514/44

(57) ABSTRACT

The present invention contemplates compositions and methods for the treatment of vascular grafts both ex vivo and in vivo. Ex vivo treatment comprises completely removing a vessel (i.e., vein or artery) from the body and treating with the compositions of the present invention. In vivo treatment comprises treating the vessel in vivo without removing the vessel completely from the body (albeit one or both ends of the vessel may be closed off in order to focus the treatment in the desired area and/or avoid systemic treatment). In one embodiment, at least a portion of the smooth muscle cells of a vessel (e.g., vein or artery) are transfected ex vivo or in vivo with a vector capable of expressing at least one phosphatase. In a preferred embodiment, smooth muscle cells are transfected with adenovirus vector comprising the gene encoding PTEN.

### GOVERNMENT SUPPORT

The development of the embodiments described herein were supported, in part, by NIH grant 1 RO1 HL072183-01, NIH grant HL03557 and NRSA 5F32H71387-2.

### FIELD OF THE INVENTION

The present invention contemplates compositions and methods for the treatment of vascular grafts both ex vivo and in vivo. At least a portion of the smooth muscle cells of a vessel (e.g., vein or artery) are transfected ex vivo or in vivo with a vector capable of expressing at least one phosphatase. In a preferred embodiment, smooth muscle cells are transfected with adenovirus vector comprising the gene encoding PTEN.

### BACKGROUND

Cardiovascular surgery is one of the most prevalent and expensive procedures in modern medicine. Coronary artery bypass grafts (CABG), also referred to as cardiac revascularizations, were performed on 519,000 Americans during the year 2000. CABG is also required for approximately 436 per million Europeans annually. Unger, F., “Cardiac Interventions In Europe 1997: Coronary Revascularization Procedures And Open Heart Surgery” Cor Europaeum 7:177-186 (1999). Other routinely performed cardiovascular operations include angioplasty (1 million/year) and percutaneous coronary interventions (1.7 million/year). Yuk-Kong, “Drug Eluting Stent: A Major Advance In Fighting Coronary Artery Stenosis” Hong Kong College Of Cardiology (2002). The initial costs of CABG procedures are greater than angioplasty ($32K versus $21K per operation) but their respective 4 year follow-up costs are nearly equivalent ($53K versus $51K). Hlatky et al., “Clinical Correlates Of The Initial And Long-Term Cost Of Coronary Bypass Surgery And Coronary Angioplasty” Am Heart J 138:376-383 (1999). Coronary surgeries, and in particular CABG, show significant failure rates primarily due to the development of neointimal hyperplasia (one year: 15-20%; 5 years: 30%; and 10 years: 50%). Follow-up CABG procedures are usually associated with a 3-5 fold increase in mortality rates over the initial operation. While the major surgery associated with CABG accounts for a high per capita expense and medical risk, more routine procedures (i.e., for example, out-patient hemodialysis) involve cardiovascular interventions that have an overall greater economic impact and carry high mortality risks.

Long-term hemodialysis treatment in the United States currently involves approximately 292,000 patients. Expectations are that each year approximately 86,000 new patients begin hemodialysis. Patel et al., “Failure Of AVF Maturation” J Vascular Surg 38:439-445 (2003). In comparison to CABG, the economic cost of hemodialysis is enormous. Hemodialysis for end-stage renal disease patients, alone, totaled $22.8 billion in the year 2001. Overall, Medicare pays approximately $53K per year for each patient requiring hemodialysis for an annual total of approximately $15.5 billion. United States Renal Data System, 2001 Annual Report, Natl Inst Health/Natl Inst Diabetes And Digestive Kidney Diseases. Similar to CABG, the development of neointimal hyperplasia is responsible for a failure to maintain patent vascular access in a high percentage of long-term hemodialysis patients.

Prevention of neointimal hyperplasia in medical procedures involving the cardiovascular system would clearly be of benefit not only to the patients themselves, but to society in general. What is needed in the art are improved compositions and methods to reduce and/or prevent the development of neointimal hyperplasia following cardiovascular procedures.

### SUMMARY OF THE INVENTION

The present invention contemplates compositions and methods for the treatment of vascular grafts both ex vivo and in vivo. Ex vivo treatment comprises completely removing a vessel (i.e., vein or artery) from the body and treating with the compositions of the present invention. In vivo treatment comprises treating the vessel in vivo without removing the vessel completely from the body (albeit one or both ends of the vessel may be closed off in order to focus the treatment in the desired area and/or avoid systemic treatment). In one embodiment, at least a portion of the smooth muscle cells of a vessel (e.g., vein or artery) are transfected ex vivo or in vivo with a vector capable of expressing at least one phosphatase. In a preferred embodiment, smooth muscle cells are transfected with adenovirus vector comprising the gene encoding PTEN.

It is not intended that the present invention be limited to the particular vector or phosphatase. However, in a preferred embodiment, an adenoviral vector comprising the human PTEN gene (or a functional portion thereof) is employed. Without limiting the invention to any particular theory of operation, it is believed that transfection of the smooth muscle cells of a vessel with such a vector, followed by the expression of PTEN, inhibits cellular proliferation, thereby reducing restenosis and/or intimal hyperplasia.

In one embodiment the present invention contemplates a method comprising: a) providing; i) a patient comprising a vein, said vein comprising smooth muscle cells; ii) an adenoviral vector comprising a nucleic acid encoding a PTEN amino acid sequence, said amino acid sequence selected from the group consisting of SEQ ID NO:1 and derivatives of SEQ ID NO:1, said derivatives comprising amino acid sequences comprising substitutions, said substitutions selected from the group consisting of Ser_Glu, Thr_Glu, Asp_Asn and Cys Ser; b) contacting said vein with said vector in situ under conditions such that said PTEN sequence is introduced into at least a portion of said smooth muscle cells to create a treated vein portion; and c) removing at least a portion of said treated vein portion from said patient to create a removed vein portion comprising a first and a second end. In one embodiment, the invention contemplates, as step b), removing at least a portion of said treated vein portion from said patient to create a removed vein portion comprising a first and a second end and, as step c), contacting said removed vein portion ex vivo with said vector under conditions such that said PTEN sequence is introduced into at least a portion of said smooth muscle cells to create a treated vein portion. In one embodiment said vein is a saphenous vein.

In one embodiment, said patient has peripheral artery disease. In one embodiment said peripheral artery disease comprises a peripheral artery having a diseased segment. In one embodiment, the method further comprises step (d) introducing said treated vein portion into said patient. In one embodiment, said introducing comprises attaching said first end of said treated vein portion to said peripheral artery distal to said diseased segment and attaching said second end of said treated vein portion to said peripheral artery proximal to said diseased segment. In one embodiment, said introducing further comprises attaching said first end of said treated vein portion to said peripheral artery under conditions such that an end-to-end anastomosis is created. In one embodiment, said introducing further comprises attaching said first end of said treated vein portion to said peripheral artery under conditions such that an end-to-side anastomosis is created. In one embodiment, said introducing further comprises attaching second end of said treated vein portion to said peripheral artery under conditions such that an end-to-end anastomosis is created. In one embodiment, said introducing further comprises attaching said second end of said treated vein portion to said peripheral artery under conditions such that an end-to-side anastomosis is created.

In one embodiment the present invention contemplates a method, comprising: a) providing; i) a patient comprising a vein (e.g., for example, a saphenous vein) and first and second arteries, said saphenous vein comprising smooth muscle cells; ii) an adenoviral vector comprising a nucleic acid encoding a PTEN amino acid sequence, said amino acid sequence selected from the group consisting of SEQ ID NO:1 and derivatives of SEQ ID NO:1, said derivatives comprising amino acid sequences comprising substitutions, said substitutions selected from the group consisting of Ser 6 Glu, Thr 6 Glu, Asp 6 Asn and Cys 6 Ser; b) removing at least a portion of said saphenous vein from said patient to create a removed vein portion, wherein said removed vein portion comprises a first end and a second end; and c) contacting said removed vein portion ex vivo with said vector under conditions such that said PTEN sequence is introduced into a least a portion of said smooth muscle cells to create a treated vein portion. In one embodiment, said amino acid sequence is proteasome-resistant. In one embodiment, said patient has cardiovascular disease. In one embodiment, said cardiovascular disease comprises coronary artery disease. In one embodiment, the method further comprises step (d) introducing said treated vein portion into said patient. In one embodiment, said introducing comprises attaching said first end of said treated vein portion to said first artery under conditions such that a distal anastomosis is created. In one embodiment, said introducing further comprises attaching said second end of said treated vein portion to said second artery under condition such that a proximal anastomosis is created. In one embodiment, said first artery comprises a coronary artery. In one embodiment, said second artery comprises the aorta.

In one embodiment, the present invention contemplates a method, comprising: a) providing; i) a patient comprising a saphenous vein, a peripheral artery and a peripheral vein, said peripheral vein comprising smooth muscle cells; ii) an adenoviral vector comprising a nucleic acid encoding a PTEN amino acid sequence, said amino acid sequence selected from the group consisting of SEQ ID NO:1 and derivatives of SEQ ID NO:1, said derivatives comprising amino acid sequences comprising substitutions, said substitutions selected from the group consisting of Ser 6 Glu, Thr 6 Glu, Asp 6 Asn and Cys 6 Ser; b) removing at least a portion of said saphenous vein from said patient to create a removed vein portion, wherein said removed vein portion comprises a first end and a second end; and c) contacting said removed vein portion ex vivo with said vector under conditions such that said PTEN sequence is introduced into a least a portion of said smooth muscle cells to create a treated vein portion. In one embodiment, said amino acid sequence is proteasome-resistant. In one embodiment, said patient has a renal disease. In one embodiment, said patient requires hemodialysis. In one embodiment, said hemodialysis comprises long-term maintenance. In one embodiment, the method further comprises (d) introducing said treated vein portion into said patient to create an arterio-venous graft. In one embodiment, said introducing comprises attaching said first end of said treated vein portion to said peripheral artery under conditions such that a first anastomosis is created. In another embodiment, said introducing further comprises attaching said second end of said treated vein portion to said peripheral vein under condition such that a second anastomosis is created. In one embodiment, said arterio-venous graft is selected from the group consisting of a wrist radiocephalic graft, a forearm radiocephalic graft and an antecubital brachiocephalic graft.

In one embodiment, the present invention contemplates a method, comprising:

a) providing; i) a patient comprising a peripheral artery and a peripheral vein, said peripheral vein comprising smooth muscle cells; ii) an adenoviral vector comprising a nucleic acid encoding a PTEN amino acid sequence, said amino acid sequence selected from the group consisting of SEQ ID NO:1 and derivatives of SEQ ID NO:1, said derivatives comprising amino acid sequences comprising substitutions, said substitutions selected from the group consisting of Ser 6 Glu, Thr 6 Glu, Asp 6 Asn and Cys 6 Ser; b) connecting said peripheral artery and said peripheral vein such that an arterio-venous fistula is created;
c) contacting said arterio-venous fistula in vivo with said vector under conditions such that said PTEN sequence is introduced into a least a portion of said smooth muscle cells to create a treated arterio-venous fistula. In one embodiment, said patient requires hemodialysis. In one embodiment, said hemodialysis comprises long-term maintenance. In one embodiment, the method further comprises prior to step (c) ligating said arterio-venous fistula. In one embodiment, said arterio-venous fistula is selected from the group consisting of a wrist radiocephalic fistula, a forearm radiocephalic fistula and an antecubital brachiocephalic fistula.

In one embodiment, the present invention contemplates a composition comprising an isolated tissue portion, said tissue portion being transfected by exposure to an adenovirus. In one embodiment, said adenovirus encodes at least a portion of a PTEN gene. In one embodiment, said tissue portion comprises a vascular tissue.

### DEFINITIONS

The terminology utilized herein is intended to be construed according to commonly used definitions known in the art, with exceptions as identified below:

The term “intimal hyperplasia”, as used herein, refers to any pathological growth of vascular smooth muscle cells after vascular trauma or injury. Further, the term also encompasses any abnormal migration of vascular smooth muscle cells from the media to the intima of vascular endothelial tissue.

The term “patient”, as used herein, refers to any mammalian organism, human or non-human.

The term “saphenous vein”, as used herein, refers to a plurality of blood vessels within a leg of a patient. Specifically, a saphenous vein may be either of two chief superficial veins of the leg: i) one originating in the foot and passing up the medial side of the leg and through the saphenous opening to join the femoral vein—called also the great saphenous vein or long saphenous vein; and ii) one originating similarly and passing up the back of the leg to join the popliteal vein at the knee—called also short saphenous vein or small saphenous vein.

The term “artery” or “arteries”, as used herein, refers to a plurality of any of the tubular branching muscular- and elastic-walled vessels that carry blood from the heart through the body within a patient.

The term “coronary artery”, as used herein, refers to either of two arteries that arise from either the left or right side of the aorta immediately above the semilunar valves and supplies oxygenated blood to the tissues of the heart itself.

The term “aorta”, as used herein, refers to any large arterial trunk that carries blood from the heart to be distributed by other arteries throughout a patient.

The term “PTEN”, as used herein, refers to an enzyme commonly referred to in the art as “phosphatase and tensin homology deleted from chromosome 10”. The human PTEN enzyme is comprised of a sequence of amino acids (i.e., for example, SEQ ID NO:2) See FIG. 14.

The term “adenovirus” or “adenoviral”, as used herein, refers to any of a family (Adenoviridae) of DNA viruses shaped like a 20-sided polyhedron. Specifically, any adenovirus used in the present invention is replication-deficient.

The term “vector”, as used herein, refers to any sequence of genetic material (i.e., for example, an adenovirus or a plasmid) into which a DNA segment has been inserted and which can be used to introduce exogenous genes into the genome of an organism, such as a patient.

The term “cardiovascular disease”, as used herein, refers to any pathological condition that reduces the proper functioning of the heart or blood vessels (i.e., for example, coronary artery disease).

The term “coronary artery disease”, as used herein, refers to any condition (i.e., for example, stenosis, restenosis, sclerosis, thrombosis etc.) that reduces the blood flow through the coronary arteries to the heart muscle.

The term “anastomosis”, as used herein, refers to any surgical union of bodily organs (especially those that are hollow and tubular). In particular, an anastomosis creates a fluid communication between or coalescence of blood vessels.

The term “vascular”, as used herein, refers to anything relating to, constituting, or affecting a tube or a system of tubes for the conveyance of a body fluid (i.e., for example, blood or lymph).

The term “smooth muscle”, as used herein, refers to any muscle tissue that lacks cross striations, that is made up of elongated spindle-shaped cells having a central nucleus, and that is found in vertebrate visceral structures (i.e., for example, the vasculature, stomach or bladder) as thin sheets performing functions not subject to conscious control (i.e., commonly referred to as involuntary muscles).

The term “fistula”, as used herein, refers to any connection between an organ, vessel, or intestine and another structure. Fistulas are usually the result of trauma or surgery, but can also result from infection or inflammation.

The term “hemodialysis” or “dialysis”, as used herein, refers to any method of removing toxic substances (impurities or wastes) from the blood when the kidneys are unable to do so. Dialysis is most frequently used for patients who have kidney failure, but may also be used to quickly remove drugs or poisons in acute situations. This technique can be life saving in people with acute or chronic kidney failure. Heniodialysis is “required” when, in the absence of hemodialysis, toxemia quickly results in death.

The term “ligate” or “ligating”, as used herein, refers to any method of restricting fluid flow in a vessel of a patient. Such a restriction may be performed by options including, but not limited to, creating two compression areas upon a vessel of a patient to create an isolated portion of the vessel. Any material or device may be used to create a compression area including, but not limited to, tying with a suture-like material or use of a clamping device.

The term “exposed” or “exposing”, as used herein, refers to a contacting of any tissue of a patient with a substance, such as a liquid solution.

The term “proteasome”, as used herein, refers to any complex of proteases responsible for targeted regulatory protein degradation (i.e., for example, the ubiquitin pathway and major histocompatibility complex antigen processing).

The term “proteasome-resistant”, as used herein, refers to any protein or enzyme having amino acid substitutions that result in a lower rate or degree (e.g., for example, 10% lower or more) of modification and/or degradation by a proteasome pathway as compared to a wild-type sequence. Such amino acid substitutions create protein or enzyme derivatives usually resulting from mutations within the nucleic acid encoding the enzyme.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell which has stably integrated foreign DNA into the genomic DNA.

The term “long-term maintenance”, as used herein, refers to any patient (whether as an in-patient or out-patient) receiving dialysis under conditions requiring a permanently implanted catheter.

### BRIEF DESCRIPTION OF THE FIGURES

FIG. 1A demonstrates exemplary data showing in vivo expression of PTEN enzyme in the rabbit kidney vasculature. FIG. 1B is a negative control. Large Arrows: medium-sized blood vessels. Small Arrows: small-sized blood vessels. Magnification: 200×.

FIG. 2 presents exemplary data showing reduced PTEN expression following either grafting or injurious trauma in vascular smooth muscle cells. Panel A: actin staining in normal rabbit: Panel B: actin staining in grafted rabbit: Panel C: PTEN staining in normal rabbit: Panel D: PTEN staining in grafted rabbit: Panel E: actin staining in normal rat. Panel F: actin staining in injured rat. Panel G: PTEN staining in normal rat: Panel H: PTEN staining in injured rat. Magnification in Panels A-D (100×); Magnification in Panels E-H (200×).

FIG. 3 presents exemplary data showing adenovirus-mediated overexpression of transgenes in canine aortocoronary saphenous vein grafts. Panel A: Xgal stain with untransfected tissue. Panel B: Xgal stain with Adâgal-transfected tissue. Panel C: Anti-PTEN stain with AdEV-transfected tissue. Panel D: Anti-PTEN stain with AdPTEN-transfected tissue. Panel E: Western immunoblot from AdPTEN-transfected tissue (lanes 1-3) and untransfected tissue (lanes 4-6).

FIG. 4 presents exemplary data of PTEN transgene expression. Panel A: Comparing PTEN expression in canine saphenous vein tissues incorporating either an adenovirus encoding PTEN (AdPTEN) or an empty adenovirus (AdEV). Panel B: Comparing PTEN expression in various canine tissues following an AdPTEN CABG procedure. Saphenous vein graft tissue (VG). Normal saphenous vein tissue (NV). Right ventricle (RV). Left ventricle (LV). +(positive control). −(negative control). Arrow—molecular weight region of PTEN.

FIG. 5 presents exemplary data showing that PTEN overexpression inhibits intimal hyperplasia. Panel A: Normal saphenous vein tissue (NV). Scale=1 mm. Panel B: Sham-transfected PBS saphenous vein graft. Panel C: AdEV-transfected saphenous vein graft tissue: Panel D: AdPTEN-transfected saphenous vein graft tissue. Arrows indicate intimal borders.

FIG. 6 presents exemplary Western blot data showing that PTEN overexpression inhibits Akt phosphorylation in canine vascular smooth muscle cells. PDGF—platelet derived growth factor.

FIG. 7 presents exemplary Western blot data showing that PTEN overexpression inhibits Akt phosphorylation in human vascular smooth muscle cells. PDGF—platelet derived growth factor.

FIG. 8 presents exemplary data showing that PTEN inhibits canine vascular smooth muscle cell proliferation. Panel A: Pulse labeling with [³H]-thymidine. Panel B: Vascular smooth muscle cell number following culture trypsinization. Open Bars: basal. Crosshatched Bars: platelet derived growth factor stimulation. Solid Bars: fetal bovine serum stimulation. Un: Sham-transfected. PTEN: AdPTEN-transfected. GFP: AdGFP-transfected.

FIG. 9 presents exemplary data showing that PTEN inhibits human vascular smooth muscle cell proliferation. Panel A: Pulse labeling with [³H]-thymidine. Panel B: Vascular smooth muscle cell number following culture trypsinization. Open Bars: basal. Crosshatched Bars: platelet derived growth factor stimulation. Solid Bars: fetal bovine serum stimulation. Un: Sham-transfected. PTEN: AdPTEN-transfected. GFP: AdGFP-transfected.

FIG. 10 presents exemplary morphological data showing that in vivo incorporation of AdPTEN inhibits rat carotid artery neointimal hyperplasia. Panel A: control (NI); Panel B: sham-transfected (PBS); Panel C: AdEV-transfected (EV); Panel D: AdPTEN-transfected (PTEN).

FIG. 11 presents a representative quantitative analysis of reduced neointimal hyperplasia by in vivo AdPTEN-incorporation in the rat carotid artery. PBS: Sham-transfected; EV: AdEV-transfected. PTEN: AdPTEN-transfected.

FIG. 12 presents representative tissue sections showing that AdPTEN-incorporation induces early medial cell apoptosis in rat carotid artery. Sections were stained with Hoecsht 33342 and visualized by fluorescence microscopy. Dashed Line—demarcation between media and adventitia. Arrows—exemplary cells having apoptotic morphological features.

FIGS. 13A & B present a representative quantitative analysis showing that AdPTEN-incorporation reduces rat carotid artery medial cell number suggesting the presence of apoptosis. Total nuclei and apoptotic nuclei were counted in Hoecsht 333421 stained vessel sections. control (NI); sham-transfected (PBS); AdEV-transfected (EV); AdPTEN-transfected (PTEN).

FIG. 13C presents a representative quantitative analysis showing that AdPTEN-incorporation reduces medial cell proliferation in rat carotid artery. sham-transfected (PBS); AdEV-transfected (EV); AdPTEN-transfected (PTEN).

FIG. 14 presents one embodiment of an amino acid sequence (SEQ ID NO:1) and corresponding nucleic acid sequence of a human PTEN enzyme (SEQ ID NO:2).

### DETAILED DESCRIPTION OF THE INVENTION

The present invention contemplates compositions and methods for the treatment of vascular grafts both ex vivo and in vivo. Ex vivo treatment comprises completely removing a vessel (i.e., vein or artery) from the body and treating with the compositions of the present invention. In vivo treatment comprises treating the vessel in vivo without removing the vessel completely from the body (albeit one or both ends of the vessel may be closed off in order to focus the treatment in the desired area and/or avoid systemic treatment). In one embodiment, a least a portion of the smooth muscle cells of a vessel (e.g., vein or artery) are transfected ex vivo or in vivo with a vector capable of expressing at least one phosphatase. In a preferred embodiment, smooth muscle cells are transfected with adenovirus vector comprising the gene encoding PTEN.

PTEN has been associated with the regulation of smooth muscle cell proliferation. Serum-stimulated DNA synthesis and Akt phosphorylation (a downstream effector of 3-phosphoinositide kinase; PIK) are reduced in the presence of PTEN. Garl et al., “Perlecan-Induced Suppression Of Smooth Muscle Cell Proliferation Is Mediated Through Increased Activity Of the Tumor Suppressor PTEN” Circ Res 94(2):175-83 (2004); Epub Dec. 1, 2003. Pathologic vascular smooth muscle cell proliferation and associated intimal hyperplasia is a known early factor that may lead to aortocoronary saphenous vein graft failure. Although it is not necessary to understand the mechanism of an invention, PIK may play a role in intimal hyperplasia via its 3-phosphoinositide lipid products, which regulate the activation of downstream effector molecules (i.e., for example, Akt and mTOR). Huang et al., “Inhibition Of Vascular Smooth Muscle Cell Proliferation, Migration, and Survival By The Tumor Suppressor Protein PTEN” Arterioscler Thromb Vasc Biol 22:745-751 (2002).

Surprisingly, very few studies have investigated PIK inhibitors' effects on vascular injury in vivo. Wortmannin, a highly selective PIK inhibitor was administered to rats prior to carotid arterial injury. Shigematsu et al., “Phosphatidylinositol 3-Kinase Signaling Is Important For Smooth Muscle Cell Replication After Arterial Injury” Arterioscler Thromb Vasc Biol 20:2373-2378 (2000). This study showed that wortmannin blocked increases in arterial Akt activation and cyclin D1 expression, and these effects correlated with inhibition of medial vascular smooth muscle cell proliferation.

### Intimal Hyperplasia And Stenosis

Intimal hyperplasia is known to be a complication of both coronary artery and peripheral artery surgical procedures and is commonly referred to as stenosis (or restenosis upon its reoccurrence). Stenting and balloon angioplasty are two surgical procedures used to physically oppose the development of intimal hyperplasia. Both stenting and balloon angioplasty require mechanical devices to hold the vascular lumen open, thereby allowing unrestricted blood flow. Stenting protocols are included in approximately 80% of balloon angioplasty procedures. Post-balloon angioplasty stenting significantly reduces the restenosis rate from 30-50% down to 20-30% when determined six months following the procedure. Likewise, post-angioplasty stenting reduces by one-half the necessity for reintervention (i.e., 10% versus 21%). Macaya et al., “Continued Benefit Of Coronary Stenting Versus Balloon Angioplasty: One-Year Clinical Follow-Up Of Benestent Trial: Benestent Study Group.” J Am Coll Cardiol. 27:255-261 (1996). If balloon angioplasty is performed alone, however, reintervention (i.e., for example, by further angioplasty or bypass surgery) is necessary in 44% of the cases within three years of the initial surgery. Editorial, “Intracoronary Stents” British Medical J 313:892-893 (1996). Despite progress in the art, the combination of balloon angioplasty with stents does not solve the problem of stenosis or restenosis development.

While it is not essential to understand the mechanism of an invention, it is believed that three major pathogenic factors comprise the phenomenon of stenosis or restenosis; i) elastic recoil; ii) negative arterial remodeling; and iii) neointimal hyperplasia. Stenting is known to effectively counteract elastic recoil and negative arterial remodeling, but post-stent neointimal hyperplasia can still cause significant restenosis. Drug-eluting stents have been introduced to treat neointimal hyperplasia-induced restenosis. A sirolimus-eluting stent (Johnson & Johnson) is reported as promising in both efficacy and safety. During a nine month trial, coronary artery surgical reinterventions were reduced from 16.8% to 4.2%; similarly, hemodialysis vascular access restenosis was reduced from 36% to 9%. Anonymous, “FDA Approves Drug-Eluting Stent For Clogged Heart Arteries” FDA News April 24 (2003).

Hemodialysis-related restenosis usually results from thrombosis and leads to a failure of the vascular access site. In fact, thrombosis-induced vascular access site dysfunction is the most common cause of hospitalization among maintenance dialysis patients. Hojs et al., “Homocysteine And Vascular Access Thrombosis In Hemodialysis Patients” Renal Failure 24:215-222 (2002). Current approaches to control vascular access failure morbidity appear unsuccessful. Vascular access related morbidity accounts for at least 25% of all hospital stays. Further, in the first year of dialysis treatment, vascular access related morbidity constitutes 50% of all patient care costs. Clearly, the incidence of vascular access failure due to thrombosis is unpredictable and creates an enormous frustration among clinicians and patients. Vascular access failure significantly reduces the overall effectiveness of dialysis and is a major factor in the relative risk of mortality for chronic dialysis patients. Hakim et al. “Hemodialysis Access Failure: A Call To Action” Kidney International 54:1029-1040 (1998).

Although it is not necessary to understand the mechanism of an invention, it is believed that vascular access failure thrombosis results from intravascular stenotic lesion formation induced by neointimal hyperplasia. Specific risk factors, however, are not defined with the exception that a synthetic graft (i.e., for example, polytetrafluoroethylene; PTFE) carries a higher risk when compared to a fully mature native arterio-venous fistula (AVF). Hojs et al., “Homocysteine And Vascular Access Thrombosis In Hemodialysis Patients” Renal Failure 24:215-222 (2002). This causal relationship between intravascular stenotic lesions and thrombi formation have lead to unsuccessful attempts to prevent stenosis. Unfortunately, the current state of the art still requires reintervention procedures within 6 months after vascular access site placement in 50-75% of hemodialysis patients.

Native AVFs provide an internal access for the hemodialysis procedure. An AVF involves the surgical joining of an artery and vein under the skin. The increased blood volume stretches the elastic vein to allow a larger volume of blood flow. The AVF requires approximately four to six weeks to heal followed by placement of a permanent catheter comprising an arterial-side port and a venous-side port. Thereafter, blood is provided to a hemodialysis machine using the arterial-side port. The hemodialysis machine, returns the blood using the venous-side port. Alternatively, an AV graft (AVG) may be used for people whose veins are not suitable for an AVF. This procedure involves surgically grafting a donor vein from the patient's own saphenous vein (in the leg), a carotid artery from a cow, or a synthetic graft from an artery to a vein. In one embodiment, the present invention contemplates arterio-venous grafts including, but not limited to, left wrist radiocephalic, left forearm radiocephalic, right wrist radiocephalic, left antecubital brachiocephalic, right antecubical brachiocephalic or a right forarm radiocephalic.

The initial creation of an AVF was performed using a radial artery and an adjacent vein. Brecia et al., “Chronic Hemodialysis Using Venipuncture And A Surgically Created Arteriovenous Fistula” N Engl J Med 275:1089-1092 (1966). Since then, fistulas between several peripheral veins and arteries have been performed including, but not limited to, left wrist radiocephalic, left forearm radiocephalic, right wrist radiocephalic, left antecubital brachiocephalic, right antecubical brachiocephalic or a right forarm radiocephalic. Lye et al., “Surgical Revision And Early Cannulation Of The Arteriovenous Fistula In Hemodialysis Patients: An Effective Technique” Hemodial Int 5:28-31 (2001).

As noted above, PTFE arterio-venous grafts are usually less effective than mature native AVFs. Patel et al., “Failure Of AVF Maturation” J Vasc Surg 38:439-445 (2003); and Simts et al., “Thrombosis Free Hemodialysis Grafts: A Possibility For The Next Century?” Seminars In Dialysis 12:44-49 (1999). Specifically, fully mature AVFs seldom form thrombi whereas PTFE grafts result in approximately 0.5-1.3 thrombotic events per patient annually. Proposed medical guidelines to place native AVFs in at least 50% of long-term hemodialysis vascular access sites are apparently going unheeded. Only 17-24% of patients in the United States have hemodialysis vascular access sites comprising native AVFs (as compared with 80% in Europe). The resistance to place native AVFs might be due to the observations that successful maturation of a native AVF is unpredictable (i.e., for example, ranging between 30%-90%). When the lower maturation rates are encountered, the functional patency of native AVFs are effectively reduced to the level of prosthetic arterio-venous grafts. Overall, it is clear that neither prosthetic (i.e., PTFE) or native AVFs prevent or sufficiently reduce stenosis-induced failure of long-term hemodialysis vascular access site patency.

One approach to reducing the failure of arterio-venous fistula stenosis and failure involves the placement of a vibrational cannula (i.e., an acoustic device) at or near the anastomosis junction. Over the period of one month, neointimal hyperplasia is expected to be reduced by 10-30%. McKenzie et al., “Methods And Kits For The Inhibition Of Hyperplasia In Vascular Fistulas And Grafts” U.S. Pat. No. 6,387,116. The present invention, however, contemplates a method comprising an ex vivo transfection of a PTEN-containing adenovirus in a native AVF prior to anastomosis with the patient's vasculature. In one embodiment, an in vivo overexpression of the transfected PTEN gene reduces and/or prevents the development of stenoic lesions and thrombosis surrounding an arterio-venous fistula.

### Reduction of Stenosis by In Vivo Protein Expression

Cell proliferation is a homeostatically balanced process required for the remodeling and healing process of mammalian tissues. Consequently, when prolonged or acute trauma is experienced by the body, cell proliferation may become overstimulated and resistant to negative feedback controls. In the vasculature, this cellular overproliferation results in stenosis and/or intimal hyperplasia. Interestingly, an equivalent physiological phenomenon is responsible for the uncontrolled growth of cancerous tumors.

Recently, a reduction in PTEN expression has been proposed as a possible marker for the development of endometrial cancer. One hypothesis for this reduced expression involves PTEN intron deletion mutations that prevent detection by various immunohistological techniques. Mutter et al., “Diagnosis Of Endometrial Precancer” U.S. Pat. No. 6,649,359 B2 (herein incorporated by reference). This unlikely commonality between the disparate cardiovascular and oncological medical fields lead to speculation that the intracellular mechanism of action of tumor suppressors may be beneficial in the reduction of stenosis.

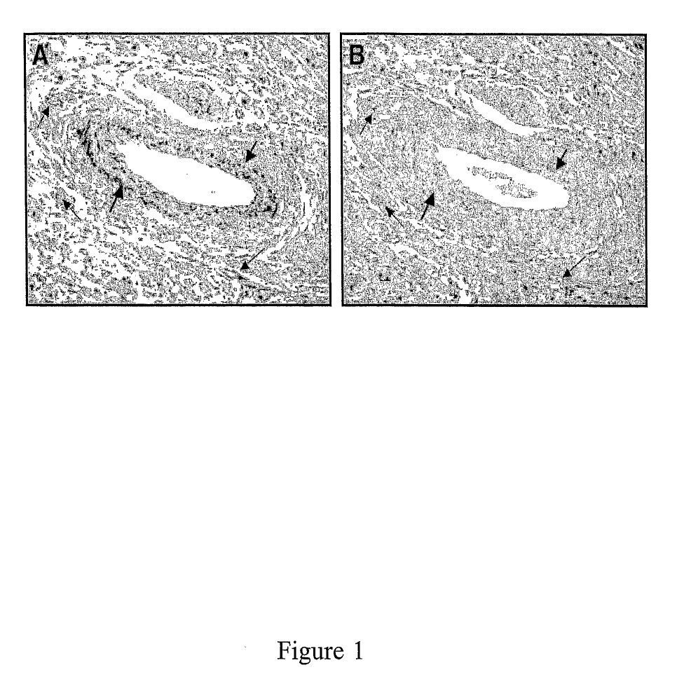

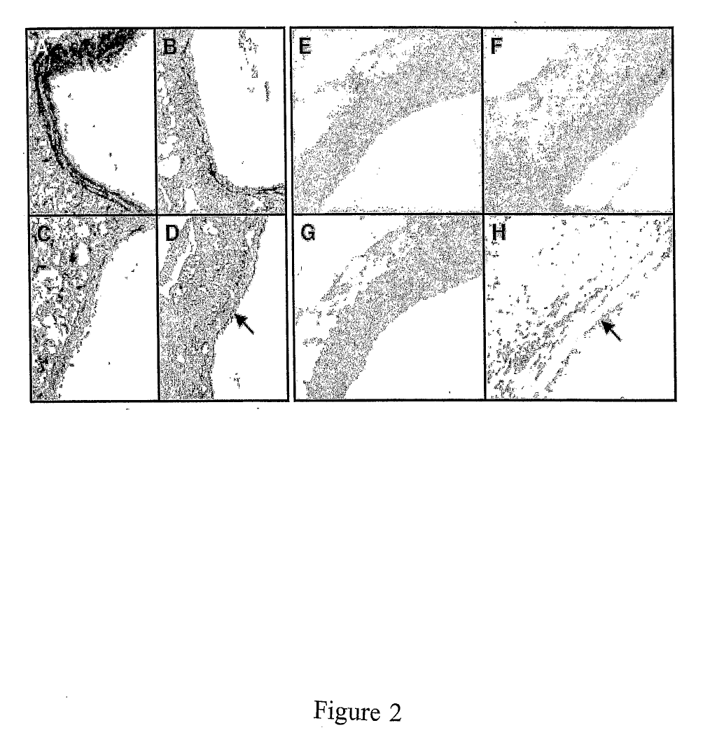

The PTEN enzyme was originally identified and studied as a tumor suppressor. The intracellular activity of PTEN regulates the phosphatidylinositol 3-kinase (PIK) cascade pathway. Deleris et al., “SHIP-2 And PTEN Are Expressed And Active In Vascular Smooth Muscle Cell Nuclei, But Only SHIP-2 Is Associated With Nuclear Speckles” J Biol Chem 278:38884-38891 (2003); Epub Jul. 7, 2003. PIK activity is known to play a role in cell growth, including cells comprising tissues of the cardiovascular system (i.e., for example, endothelial tissue, coronary tissue, venous tissue, arterial tissue etc.). One embodiment of the present invention contemplates expression of PTEN in vascular smooth muscle cells. See FIG. 1. In another embodiment, the present invention contemplates decreased activity of PTEN in wild-type vascular smooth muscle cells in the presence of injury or trauma. See FIG. 2. Although it is not necessary to understand the mechanism of an invention, it is believed reductions in intracellular PTEN increases PIK signaling that stimulates cellular growth, thereby resulting in the development of neointimal hyperplasia.

Neointimal hyperplasia contributes to the progressive occlusion of both saphenous vein grafts after CABG procedures and AVF placements. The failure of either CABG or AVF results in substantial patient morbidity. The present invention contemplates compositions and methods to reduce and/or prevent neointimal hyperplasia comprising a recombinant adenovirus encoding PTEN. In one embodiment, the recombinant adenovirus encoding PTEN is transfected into a saphenous vein graft. In another embodiment, the recombinant adenovirus encoding PTEN is transfected into an arterio-venous fistula. Although it, is not necessary to understand the mechanism of an invention, it is believed that overexpression of transfected PTEN inhibits PIK signaling and subsequent cell growth, thereby reducing and/or preventing neointimal hyperplasia (i.e., for example, stenosis or restenosis).

Incorporation of a nucleic acid, such as an adenovirus vector, into a host tissue may be performed using an ex vivo embodiment of the present invention. This embodiment carries significant advantages over other current methods in the field of gene therapy such as, targeted immunotherapy and stem cell incorporation which typically involve systemic adenoviral delivery. Bartel et al., “MMSC1-An MMAC1 Interacting Protein” U.S. Pat. No. 6,337,192 B1 (herein incorporated by reference). One embodiment of the present invention avoids systemic adenovirus vector delivery by ex vivo transfection of a vector during a surgical procedure. During pre-implantation preparations, a tissue may be incubated for a predetermined period of time in a buffer solution containing an adenovirus vector under conditions that the vector is absorbed by the tissue. Chiu-Pinheiro et al., “Gene Transfer To Coronary Bypass Conduits” Ann Thorac Surg 74:1161-1166 (2002). Following grafting (i.e., for example, by an anastomosis) of the incubated tissue within the body, and after an appropriate isolation time, the tissue graft expresses any encoded protein(s) or enzyme(s) comprising the transfected vector that are operably linked to a promoter. Clearly, the expressed protein(s) or enzyme(s) will have a localized effect due to their intracellular site of expression and subsequent site of action. In one embodiment of the present invention the adenoviruses are replication-deficient (i.e., for example, “gutted”, wherein the adenoviruses lack all adenoviral coding regions).

The present invention contemplates adenoviral vectors coexpressing critical viral gene functions in HEK 293 cell lines. The present invention contemplates isolation and characterization of HEK 293 cell lines capable of constitutively expressing the adenoviral polymerase protein. In addition, the present invention contemplates the isolation of HEK 293 cells which not only express the E1 and polymerase proteins, but also the adenoviral-preterminal protein. Isolation of cell lines coexpressing the E1, adenovirus polymerase and preterminal proteins demonstrate that three genes critical to the life cycle of adenvirus can be constitutively coexpressed, without toxicity. Chamberlain et al. “Adenovirus Vectors” U.S. Pat. No. 6,057,158 (2000)(herein incorporated by reference).

In order to delete critical genes from self-propagating adenoviral vectors, proteins encoded by the targeted genes have to first be coexpressed in HEK 293 cells along with the E1 proteins. Therefore, only those proteins which are non-toxic when coexpressed constitutively (or toxic proteins inducibly-expressed) can be utilized. Coexpression in HEK 293 cells of the E1 and E4 genes has been demonstrated (utilizing inducible, not constitutive, promoters). Yeh et al, J. Virol. 70:559 (1996); Wang et al. Gene Therapy 2:775 (1995); and Gorziglia et al., J. Virol. 70:4173 (1996). The E1 and protein IX genes (a virion structural protein) have been coexpressed, and coexpression of the E1, E4, and protein IX genes has also been described. Caravokyri et al. Virol. 69:6627 (1995); and Krougliak et al., Hum. Gene Ther. 6:1575 (1995).

The present invention contemplates cell lines coexpressing E1 and E2b gene products. The E2b region encodes the viral replication proteins which are absolutely required for adenoviral genome replication. Doerfler, supra and Pronk et al., Chromosoma 102:S39-S45 (1992). The present invention provides 293 cells which constitutively express the 140 kD adenoviral polymerase. The isolation of 293 cells which express the adenoviral preterminal protein utilizing an inducible promoter has been reported. Schaack et al., J. Virol. 69:4079 (1995). The present invention contemplates a high-level, constitutive coexpression of the E1, polymerase, and preterminal proteins in HEK 293 cells, without toxicity. These novel cell lines permit the propagation of novel adenoviral vectors deleted for the E1, polymerase, and preterminal proteins.

One embodiment of the present invention contemplates a method for clinical vascular gene therapy comprising ex vivo transfection with adenovirus vectors. In one embodiment, an adenovirus vector comprises at least a portion of the PTEN gene (AdPTEN). In one embodiment, AdPTEN transfection reduces intimal hyperplasia for at least ninety days. This is much greater than the typical duration of less than three weeks for transgene expression using first-generation adenovirus vectors. Channon et al., “Efficient Adenoviral Gene Transfer To Early Venous Bypass Grafts: Comparison With Native Vessels” Cardiovas Res 35:505-513 (1997). Although it is not necessary to understand the mechanism of an invention, it is believed that a temporary inhibition of proliferation triggering mechanisms (i.e., for example, PIK) early after vascular injury may be sufficient to produce intermediate or long-term effects. Further, it is believed that inhibition of vascular smooth muscle cell proliferation may only be required until the damaged vessel endothelium is regenerated. Consequently, adenoviral vectors may be ideal for clinical vascular gene therapy, as their limited length of action would avoid the possibility of long-term toxicity. In one embodiment, the present invention contemplates a transgene comprising a pro-apoptotic enzyme (i.e., for example, PTEN) delivered by a replication-deficient adenovirus. In one embodiment, the transgene is delivered to harvested or artificial vascular conduits prior to implantation.

### Adenovirus Vectors Encoding Protease-Stable PTEN

The PTEN enzyme has been shown to be degraded by the proteasome. Torres et al., “The Tumor Suppressor PTEN Is Phosphorylated By The Protein Kinase CK2 At Its C Terminus: Implications For PTEN Stability To Proteasome-Mediated Degradation.” J Biol Chem 276:993-998 (2001); Vazquez et al., “Phosphorylation Of The PTEN Tail Regulates Protein Stability And Function.” Mol Cell Biol 20:5010-5018 (2000); and Vazquez et al., “Phosphorylation Of The PTEN Tail Acts As An Inhibitory Switch By Preventing Its Recruitment Into A Protein Complex” J Biol Chem 276:48627-48630 (2001). Proteasome-mediated PTEN degradation may be responsible for the observed loss of PTEN expression following vascular injury. See Example 2. In one embodiment, the present invention contemplates PTEN compositions that are resistant to proteasome degradation. In one embodiment, a proteasome-resistant PTEN enzyme comprises at least one mutation in a PTEN-coding region. In one embodiment, a proteasome-resistant PTEN enzyme improves PTEN-mediated reductions of stenosis.

A nucleic acid comprising a mutated gene coding for a proteasome-resistant PTEN enzyme may also be transfected into a modified adenoviral delivery vector. PTEN is highly homologous across species, in regards to both DNA and protein sequences. In one embodiment, a polymerase chain reaction amplification is modified to achieve specificity for an adenoviral PTEN transgene. In one embodiment, an amplification method for an adenoviral PTEN transgene provides a forward primer specific for an adenovirus promoter and a reverse primer specific for a human PTEN sequence. In one embodiment, the vector comprises a constitutive cytomegalovirus promoter.

In another embodiment, the modified adenoviral delivery vector comprises a vascular smooth muscle-specific promoter. In one embodiment, the promoter comprises an SM22á promoter wherein said promoter directs protein expression in vascular smooth muscle cells. Akyurek et al., “SM22á Promoter Targets Gene Expression To Vascular Smooth Muscle Cells In Vitro And In Vivo” Mol Med 6:983-991 (2000); Li et al., “Expression of The SM22á Promoter In Transgenic Mice Provides Evidence For Distinct Transcriptional Regulatory Programs In Vascular And Visceral Smooth Muscle Cells” J Cell Biol 132:849-859 (1996); and Frame et al., “Localized Adenovirus-Mediated Gene Transfer Into Vascular Smooth Muscle In The Hamster Cheek Pouch” Microcirculation 8:403-413 (2001). Use of the SM22á promoter has been disclosed to support systemic delivery of adenovirus vectors consisting of heterologous genes that control the cell cycle (i.e., retinoblastoma gene, p53, cell cycle regulatory kinase, CDK kinase, cyclins, cell cycle regulatory proteins, angiogenesis gene). Expression of these gene families affect cell proliferation and inhibit restenosis following balloon angioplasty and arterial injury or stimulate angiogenesis following placement of bioprosthetic grafts or stents. Parmacek et al., “Promoter Smooth Muscle Cell Expression” U.S. Pat. No. 6,331,527 (herein incorporated by reference).

A number of stabilizing and/or activating PTEN mutations have been previously described, primarily with respect to cancer. As a tumor suppressor, PTEN (TS10Q23.3) mutations may result in enzyme inactivation thereby allowing precancerous growths to develop into tumors. Consequently, specific PTEN mutations are identified as probable causes of some cancers. Steck et al., “Tumor Suppressor Designated TS10Q23.3” U.S. Pat. No. 6,482,795. (herein incorporated by reference). The '795 patent discloses several types of adenoviral vectors encoding mutants of the PTEN enzyme that might be capable of supporting ex vivo treatment of bone marrow cells in an effort to reduce tumor growth following systemic reintroduction to a patient. The present invention contemplates integrating these mutations into one embodiment of an adenovirus vector encoding a PTEN enzyme. Although it is not necessary to understand the mechanism of an invention, it is believed that these stabilizing and/or activating PTEN mutations will enhance stability of the overexpressed PTEN enzyme following transfection into vein grafts or other targets. One suspected target domain for the proteasome is the PTEN carboxyl terminus which comprises a PEST domain. Lee et al., “Crystal Structure Of The PTEN Tumor Suppressor; Implications For Its Phosphoinositide Phosphatase Activity And Membrane Association.” Cell 99:323-334 (1999); Rechsteiner et al., “PEST Sequences And Regulation By Proteolysis.” Trends In Biochem Sci 21:267-271 (1996).

The PTEN carboxyl terminus has also been found to be phosphorylated on several serine and threonine residues, which may result in the direct translocation of the PTEN enzyme to the plasma membrane. It is suspected that PTEN phosphatase activity directed to the 3-phosphoinositides occurs at the plasma membrane. Modification in PTEN serine/threonine phosphorylation is suspected of imparting increased stability to proteasome activity. Further, it has been suggested that PTEN phosphorylation is associated with decreased PTEN activity while PTEN dephosphorylation is associated with increased PTEN activity. Torres et al., “Phosphorylation-Regulated Cleavage Of The Tumor Suppressor PTEN By Capsase-3: Implications For The Control Of Protein Stability And PTEN-Protein Interactions” J Biol Chem 278:30652-30660 (2003). Specific PTEN enzyme mutations are known to improve proteasome stability. See Table 1. One of skill in the art will recognize that serine or threonine substitutions with glutamate may also be accomplished by substitution with aspartate. Similarly, it is also obvious to one of skill in the art that multiple mutations may provide improved stability over a single mutation. In fact, this hypothesis has already been tested. Torres et al. (2001); and Lee et al.

TABLE 1
PTEN Enzyme Mutations That Alter Stability And/Or Activity

| Mutation | Effect | Reference |
|---|---|---|
| Position 370: Ser 6 Glu | Stabilizing | Torres et al. (2001) |
| Position 380: Ser 6 Glu | Stabilizing | Torres et al. (2001); |
| | | Vazquez et al. (2000) |
| Position 382: Thr 6 Glu | Stabilizing | Torres et al. (2001); |
| | | Vazquez et al. (2000) |
| Position 383: Thr 6 Glu | Stabilizing | Torres et al. (2001) |
| Position 385: Ser 6 Glu | Stabilizing | Torres et al. (2001) |
| Position 301: Asp 6 Asn | Stabilizing | Torres et al. (2003) |
| Position 371: Asp 6 Asn | Stabilizing | Torres et al. (2003) |
| Position 375: Asp 6 Asn | Stabilizing | Torres et al. (2003) |
| Position 384: Asp 6 Asn | Stabilizing | Torres et al. (2003) |
| Position 071: Cys 6 Ser | Activating | Lee et al. |

One embodiment of the present invention contemplates at least one mutation in the PTEN coding region that improves PTEN stability and stenosis reduction efficacy in vascular smooth muscle cells following a vein grafting procedure. Such mutations may result in amino acid substitutions within the PTEN primary amino acid sequence, thus creating derivatives. The present invention contemplates derivatives of SEQ ID NO:1 having amino acid substitutions as defined in Table 1, wherein the derivatives are proteasome-resistant.

### Saphenous Vein Grafts

Saphenous vein grafts (SVGs) represent the most common conduit used for surgical revascularization procedures, including coronary artery bypass grafting (CABG). Unfortunately, long-term aortocoronary SVG efficacy is limited by intimal hyperplasia (IH) and subsequent accelerated atherosclerosis, resulting in a 10-year graft failure rate approaching 50%. Campeau et al., “Atherosclerosis And Late Closure Of Aortocoronary Saphenous Vein Grafts: Sequential Angiographic Studies At 2 Weeks, 1 Year, 5 to 7 Years, and 10 to 12 Years After Surgery” Circulation 68(3 Pt 2):II1-II7 (1983); Bourassa et al., “Changes In Grafts And Coronary Arteries After Saphenous Vein Aortocoronary Bypass Surgery: Results At Repeat Angiography” Circulation 65(7 Pt 2):90-97 (1982). Current attempts to limit SVG stenosis include technical considerations, anti-platelet therapy and lipid-lowering medications. Souza et al., “Improved Patency In Vein Grafts Harvested With Surrounding Tissue: Results Of A Randomized Study Using Three Harvesting Techniques” Ann Thorac Surg 73:1189-1195 (2002); Goldman et al., “Improvement In Early Saphenous Vein Graft Patency After Coronary Bypass Surgery With Antiplatelet Therapy Results Of A Veterans Administration Cooperative Study” Circulation 77:1324-1332 (1988). Despite these efforts, SVG failure after CABG remains a difficult problem leading to recurrent angina and a 10-15% incidence of reintervention CABG surgery. Czerny et al, “Coronary Reoperations: Recurrence Of Angina And Clinical Outcome With And Without Cardiopulmonary Bypass” Ann Thor Surg 75:847-852 (2003).

Intimal hyperplasia begins early after vein graft implantation and can eventually lead to luminal stenosis and occlusion. Intimal hyperplasia is characterized by abnormal migration of vascular smooth muscle cells from the media to the intima. Vascular smooth muscle cells subsequently proliferate and undergo hypertrophy, with associated deposition of an extracellular connective tissue matrix. Gibbons et al, “The Emerging Concept Of Vascular Remodeling” New Engl J Med 330:1431-1438 (1994). Although not completely characterized, these pathological changes are caused by the release of mitogenic growth factors in the setting of vascular injury. Braun-Dullaeus et al., “Cell Cycle Progression: New Therapeutic Target For Vascular Proliferative Disease” Circulation 98:82-89 (1998). Many growth factors and hormones may trigger intimal hyperplasia by activating PIK in vascular smooth muscle cells. PIK is a lipid kinase that phosphorylates phosphatidylinositol at the D-3 position of the inositol ring, and the resulting products, phosphatidylinositol 3,4-bisphosphate and phosphatidylinositol 3,4,5-trisphosphate are potent signaling molecules that are known to regulate cell proliferation, migration and survival. Furman et al., “Phosphoinositide Kinases” Annu Rev Biochecm 67:481-507 (1998); Rameh et al., “The Role Of Phosphoinositide 3-Kinase Lipid Products In Cell Function” J Biol Chem 274:8347-8350 (1999). Recently, the drug sirolimus (rapamycin), which inhibits a downstream effector of PIK (i.e., for example, mammalian target of rapamycin, or mTOR), has been reported in studies to reduce in-stent stenosis. Sousa et al., “Sustained Suppression Of Neointimal Proliferation By Sirolimus-Eluting Stents: One-Year Angiographic And Intravascular Ultrasound Follow-Up” Circulation 104:2007-2011 (2001). In addition to mTOR, however, PIK activates many other effectors, such as Akt, which associates with membrane-bound 3-phosphoinositides and has been implemented as a putative mediator of cell growth, proliferation and survival. Downward J., “Mechanisms And Consequences Of Activation Of Protein Kinase B/Akt” Curr Opin Cell Biol 10:262-267 (1998). Consequently, PIK is a potential upstream regulator of cellular proliferation whose inhibition might produce inhibitory effects on the development of intimal hyperplasia. A systemic administration to inhibit the PIK cascade by phosphatases (i.e., for example, PTEN) may result in serious clinical toxicity, potentially expressing the side effects observed with traditional chemotherapeutic drugs, such as rapamycin. In contrast, the present invention contemplates an embodiment that locally (i.e., for example, intracellularly) targets the phospholipid products of the PIK cascade, thereby specifically making the effects of PTEN proximal to PIK signalling pathway downstream effectors, such as mTOR and Akt. In one embodiment, PTEN overexpression proximally inhibits the entire cascade of downstream PIK effectors, thereby providing a more potent inhibitory effect on intimal hyperplasia than a specific inhibition of any single downstream effector.

Putative inhibitors of vascular smooth muscle cell PIK activity comprise endogenous enzymes. For example, the phosphoinositide signaling system may be inhibited by the dephosphorylation of phosphatidylinositol 3,4-bisphosphate and phosphatidylinositol 3,4,5-trisphosphate. In one embodiment, an endogenous enzyme dephosphorylates phosphatidylinositol 3,4-bisphosphate and phosphatidylinositol 3,4,5-trisphosphate. In one embodiment, a dephosphorylating enzyme comprises ‘phosphatase and tensin homolog on chroinosome 10’ (PTEN). Although it is not necessary to understand the mechanism of an invention, it is believed that PTEN hydrolyzes the 3-phosphoinositide lipid products of PIK including, but not limited to, phosphatidylinositol 3,4-bisphosphate and phosphatidylinositol 3,4,5-trisphosphate. Further, it is believed that 3-phosphoinositide lipid dephosphorylation prevents downstream activation of PIK effector molecules. For example, adenovirus-mediated expression of PTEN in rabbit vascular smooth muscle cells is known to inhibit platelet derived growth factor induced cell proliferation, migration and survival. Haung et al., “Inhibition Of Vascular Smooth Muscle Cell Proliferation, Migration And Survival By The Tumor Suppressor Protein PTEN” Arterioscler Thromb Vasc Biol 22:745-751 (2002). Smooth muscle growth factors (i.e., for example, platelet derived growth factor and fetal bovine serum) are powerful vascular smooth muscle cell mitogens and survival factors. In particular, platelet derived growth factor is known to be released during arterial injury and contributes to the development of intimal hyperplasia. Walker et al., “Production Of Platelet-Derived Growth Factor-Like Molecules By Cultured Arterial Smooth Muscle Cells Accompanies Proliferation After Arterial Injury” Procd Natl Acad Sci USA 83:7311-7315 (1986); Nabel et al., “Recombinant Platelet-Derived Growth Factor B Gene Expression In Porcine Arteries Induce Intimal Hyperplasia In Vivo” J Clin Invest 91:1822-1829 (1993); and Heldin et al., “Mechanism And In Vivo Role Of Platelet-Derived Growth Factor” Physiol Rev 79:1283-1316 (1999).

A canine model of aorotocoronary saphenous vein graft intimal hyperplasia has been developed. Brody et al., “Histologic Fate Of The Venous Coronary Artery Bypass In Dogs” Am J Pathol 66:111-130 (1972); Brody et al., “Changes In Vein Grafts Following Aorto-Coronary Bypass Induced By Pressure And Ischemia” J Thorac Cardiovasc Surg 66:847-853 (1972); and Silver et al. “Aortocomary Bypass Graft In Dogs: Late Histological Consequences” Pathology 8:343-351 (1976). Histologic changes are observed in these canine models that closely resemble consequences of human saphenous vein grafts, including but not limited to, medial fibrosis and intimal hypertrophy (i.e., usually occurring within one month post-surgery). At three months post-surgery, follow-up studies indicated that the intimal area and the intima/media ratio were increased in saphenous vein graft tissue. Petrofski et al., “Gene Delivery To Aortocoronary Saphenous Vein Grafts In A Large Animal Model Of Intimal Hyperplasia” J Thorac Cardiovas Surg 127:27-33 (2004).

### EXPERIMENTAL

The following examples are included only to demonstrate specific embodiments of the invention. It should be appreciated by those of skill in the art that the techniques disclosed in these examples represent techniques identified by the inventor to function well in the overall practice of the invention. However, those of skill in the art should, in the light of the present disclosure, appreciate that many changes can be made in these specific embodiments which are disclosed and still obtain a like or similar result without departing from the spirit and scope of the invention.

### Example 1

### Determination of In Vivo PTEN Expression in Vascular Smooth Muscle Cells

This example demonstrates that the PTEN enzyme is naturally expressed in vascular smooth muscle cells.

Rabbit kidneys were: i) sectioned; ii) fixed with paraformaldehyde; iii) embedded in paraffin, and iv) stained with a mouse PTEN monoclonal antibody (clone A2B1, Santa Cruz Biotechnology, Santa Cruz Calif.). The PTEN monoclonal antibody was detected with the addition of an anti-mouse antibody conjugated to horseradish peroxidase. Thereafter, the sections were counterstained with hematoxylin.

The results show that PTEN is most highly expressed in the vascular smooth muscle cells of blood vessels, including both medium-sized vessels (large arrows) and small-sized vessels (small arrows). See FIG. 1A. A negative control was performed in the absence of mouse PTEN monoclonal antibody. No staining was observed for either medium-sized vessels (large arrows) or small-sized vessels (small arrows) in these serial sections. See FIG. 1B.

These data conclusively show that PTEN is a naturally expressed enzyme in vascular smooth muscle cells.

### Example 2

### Loss of In Vivo PTEN Expression in Vascular Smooth Muscle Cells after Injury

This example demonstrates that the natural expression PTEN enzyme is reduced following vascular injury induced by either grafting or trauma.

Anesthetized rabbits were subjected to jugular vein-carotid interposition grafting. Three days later, normal (See FIG. 2 Panels A & C) and grafted (See FIG. 2 Panels B & D) vessels were harvested, sectioned, fixed in paraformaldehyde and embedded in paraffin. Tissue sections shown in Panels A & B were stained with anti-smooth muscle actin (clone HHG-35) while Panels C & D were stained with anti-PTEN. Both antibodies were detected according to the method described in Example 1.

Expression of actin and PTEN was readily detectable in normal vessels. After vein grafting, actin staining was reduced (due to the thinning of the vessel walls) but still remained intense. In contrast, PTEN expression was almost absent after grafting (See FIG. 2 Panel D, arrow). Negative controls (not shown) did not stain for either actin or PTEN.

The expression of actin and PTEN subsequent to carotid arterial injurious trauma was studied in anesthetized rats. Three days following injury, uninjured (See FIG. 2 Panels E & G) and injured (See FIG. 2 Panels F & H) vessels were prepared as above, with the exception that antibody detection was performed with alkaline phosphatase-conjugated secondary antibody. Both actin and PTEN were readily detectable in normal, uninjured arteries. In the injured vessels, actin staining remained intense (despite thinning of the vessel walls) whereas PTEN expression was markedly reduced (See FIG. 2 Panel H, arrow). Negative controls (not shown) did not stain for either actin or PTEN.

These data clearly show that PTEN expression is reduced following vascular trauma (i.e., by surgical grafting procedures or injury).

### Example 3

### Construction of a Recombinant Adenovirus Encoding a PTEN Enzyme

This example presents one protocol which results in an adenovirus vector capable of ex vivo transfection into a host chromosome. He et al., “A Simplified System For Generating Recombinant Adenoviruses” Proc Natl Acad Sci USA 95:2509-2514 (1998). This particular embodiment utilizes a recombinant, replication-deficient adenovirus directing the expression of wild-type human PTEN (ADPTEN) as previously described. Huang et al., “PTEN Modulates Vascular Endothelial Growth Factor Mediated Signaling And Angiogenic Effects” J Biol Chem 277:127:27-33 (2002). The procedures and handling of animals and tissues exposed to recombinant adenovirus were approved by the Institutional Biosafety Committee of Duke University in compliance with guidelines from the NIH.

A full-length human PTEN cDNA was excised from the starting plasmid pcDNA3 and ligated into the vector pShuttle-CMV? (Stratagene, La Jolla Calif.). Takayama et al., “Expression And Location Of Hsp70/Hsc-Binding Anti-Apoptotic Protein BAG-1 And Its Variants In Normal Tissues And Tumor Cell Lines” Cancer Research 58:3116-3131 (1998); and Froesch et al., “BAG-1L Protein Enhances Androgen Receptor Function” J Biol Chem 273:11660-11666 (1998). The resultant plasmid was linearized with PmeI and co-transformed with the plasmid pAdEasy-1 ? (Stratagene, La Jolla Calif.) into BJ5183 E. coli by electroporation to allow homologous recombination with pAdEasy-1?. Recombinant plasmids were identified by a characteristic restriction digestion pattern following digestion with PacI, thereby creating a large-scale plasmid preparation. This plasmid DNA was linearized with PacI and transfected in serum-free medium into HEK-293 cells (i.e., a human embryonic kidney cell culture) in a T25 flask using Lipofectamine-Plus? transfection reagent (Invitrogen Life Technologies, Carlsbad Calif.). After 3 hours, the medium was changed to Dulbecco's modified Eagle medium (DMEM; Invitrogen Life Technologies, Carlsbad, Calif.) containing 10% fetal bovine serum (FBS) and 1% penicillin-streptomycin (pen-strep). After 7-10 days, plaques were observed in the cell monolayer, indicating virus replication. The cells were harvested by scraping them from the flask, pelleted and resuspended in 2 ml phosphate buffered saline (PBS). This cell mixture was subjected to three rounds of freeze-thaw in liquid nitrogen to lyse the cells which released the recombinant PTEN encoded adenoviruses. One-half of the mixture (i.e., one milliliter) was then applied to a T75 flask of HEK-293 cells in DMEM, 2% FBS, 1% pen-strep and the cells were incubated at 37° C. until plaques were observed; usually occurring after 3-5 days of incubation. The cells were harvested as described above, and this process was repeated 2-3 times to amplify the adenovirus.

Verification of adenovirus replication was performed by applying dilutions of virus-containing cell lysate to HEK-293 cell cultures. After incubation overnight at 37° C. in DMEM/2% FBS the HEK-293 cells were lysed in a Triton? lysis buffer. An aliquot of each cell lysate was separated by SDS-polyacrylamide gel electrophoresis (PAGE) and transferred to nitrocellulose. Western blots were then formed with a mouse monoclonal PTEN antibody (clone A2B1; Santa Cruz Biotechnology, Santa Cruz CA).

The PTEN-encoding adenovirus vector (AdPTEN) was found to direct high-level expression of PTEN protein in target cells. For example, an aliquot of crude virus-containing cell lysate was used to infect fifty 15-cm dishes of HEK-293 cells in a large-scale adenovirus preparation. After 2-3 days, when plaques were readily apparent, the cells were harvested by scraping and pelleted by centrifugation. The cell pellet was disrupted by sonication and the released virus was purified by ultracentrifugation on a CsCl density gradient (1.3/1.4 g/ml). Purified adenovirus was diluted in virus storage buffer and 10 ìl aliquots were stored at −80° C. Virus titer was determined by spectrophotometry by measuring optical density between 260-280 nanometer wavelengths.

Control viruses were also constructed, including an empty adenovirus containing no cDNA insert (AdEV), and adenoviruses containing the coding sequence for â-galactosidase (Adâgal) or a green fluorescent protein (AdGPF).

### Example 4

### Transfection of AdPTEN into Canine Saphenous Vein During CABG Surgery

This example presents data showing that an ex vivo transfection of AdPTEN into a saphenous vein graft prior to anastomosis with a coronary artery. Petrofski et al., “Gene Delivery To Aortocoronary Saphenous Vein Grafts In A Large Animal Model Of Intimal Hyperplasia” J Thorac Cardiovasc Surg 127:27-33 (2004).

Prior to the surgery, the canines were sedated, intubated and heparinized (100 U/kg) and a one milliliter AdPTEN PBS solution containing 5×10¹¹ total virus particles was prepared. First, approximately 10 centimeters of saphenous vein was harvested from one hindleg of each animal. Both ends of the saphenous vein were then ligated and the AdPTEN PBS solution was injected into the lumen of the saphenous vein for 20-30 minutes until a distension pressure of approximately 10 mm Hg was obtained.

During the AdPTEN incubation with the saphenous vein, a partial lower sternotomy was performed on the animal. Prior to grafting the AdPTEN-containing saphenous vein, the lumen was flushed clear of AdPTEN with fresh AdPTEN-free PBS. The AdPTEN-containing saphenous vein was attached to the ascending aorta by an end-to-side anastomosis. Using a myocardial stabilizer (Guidant, Indianapolis, Ind.) an end-to-side anastomosis was then performed between the saphenous vein graft and the distal left anterior descending coronary artery. The proximal left anterior descending coronary artery was subsequently ligated, rendering the anterior left ventricle dependent upon flow through the saphenous vein graft (i.e., implementing the functional purpose of the CABG procedure). Immediately after completion of the distal anastomosis, adequate blood flow ($ 25 ml/min) through the saphenous vein graft was confirmed by an ultrasonic vascular probe (Transonic Systems, San Diego Calif.). During recovery, the animals were maintained on buffered aspirin (325 mg/day).

### Example 5

### Reduction of Stenosis Following AdPTEN Transfection into Saphenous Vein Grafts

The following experiment presents data showing that in vivo expression of PTEN reduces post-surgical stenosis.

Thirty-six mongrel dogs (27-32 kg) underwent CABG procedures according to Example 4. These animals were divided into following groups: AdPTEN Group: 12 animals receiving vein grafts transfected with an adenovirus vector encoding PTEN. AdEV Group: 12 animals receiving vein grafts transfected with an empty adenovirus vector. PBS Group: 9 animals receiving vein grafts sham-transfected with phosphate buffered saline. Adâgal Group: 3 animals receiving vein grafts transfected with an adenovirus vector encoding â-galactosidase. All animal procedures were humanely performed in accordance with regulations adopted by the National Institutes of Health (NIH) and approved by the Animal Care and Use Committee of Duke University.

At thirty and ninety days after the CABG procedure all animals underwent coronary arteriography via the femoral artery. A 6F coronary catheter was placed into the left coronary artery under fluoroscopic guidance, and radiopaque dye was used to confirm patency of the saphenous vein graft. Extensive filling defects consistent graft wall thickening were noted throughout the PBS and AdEV-transfected saphenous vein grafts. AdPTEN-treated saphenous grafts did not demonstrate these severe defects.

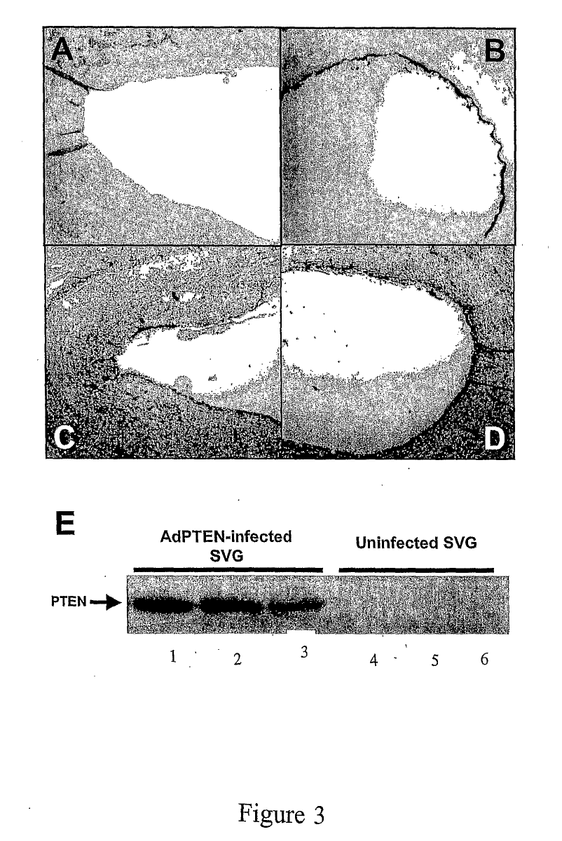

Transgene expression was confirmed by histologic staining (see Example 6) in Adâgal- and AdPTEN-treated saphenous vein grafts that were explanted three days following the CABG procedure. See FIG. 3A-D. Transgene expression was robust and circumferential in the intima, with more diffuse expression throughout the media.

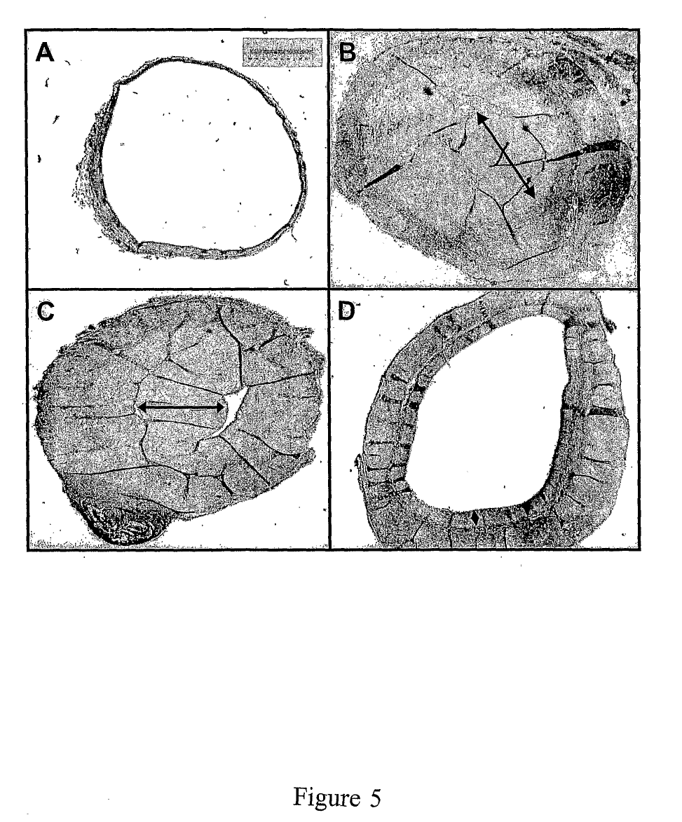

AdPTEN-transfected saphenous vein grafts demonstrated a reduced intimal area as compared to AdEV-transfected saphenous vein grafts and sham-transfected PBS control vein grafts (1.39″0.11 mm; 2.35″0.3 mm and 2.57″0.4 mm, respectively). See FIG. 5. Additionally, the intimal/media ratio was lower in AdPTEN-transfected saphenous vein grafts as compared to AdEV-transfected saphenous vein grafts and sham-transfected PBS control vein grafts (0.5″0.05; 1.43″0.18 and 1.11″0.14, respectively). Medial area and maximum/minimum wall thicknesses were not significantly different among groups.

### Example 6

### Histological Examination of PTEN Overexpression

This example demonstrates that the transfection of AdPTEN into a saphenous vein graft is capable of in vivo PTEN overexpression.

Tissues were collected from animals undergoing CABG according to Example 5, including samples of the saphenous vein graft, non-grafted saphenous vein, left and right ventricle, liver and lung. Saphenous vein segments were either frozen or embedded in paraffin and cut into 10 ìm sections. Hematoxylin/eosin (H&E) and X-gal staining were performed by standard techniques. Shah et al., “Adenovirus-Mediated Genetic Manipulation Of The Myocardial â-Adrenergic Signaling System In Transplanted Hearts” J Thorac Cardiovasc Surg 123:581-588 (2000). An anti-PTEN monoclonal antibody (clone A2B1), Santa Cruz Biotechnology, Santa Cruz Calif.) was used to immunostain for PTEN expression.

In three of the animals, short segments of AdPTEN-transfected saphenous vein graft tissues were cultured for three days and PTEN expression assessed by Western immunoblotting according to the procedures outlined in Example 8. In saphenous veins undergoing adenoviral transfection, Western blotting revealed marked PTEN expression compared to untransfected saphenous veins. See FIG. 3E.

5-ìm transverse vessel sections were H&E stained and measurements made using Image Tool v.3.0 (The University Of Texas Health Sciences Center, San Antonio Tex.) as previously described. Petrofski et al. (2004). For each animal, three sections from each third of the saphenous vein graft were analyzed (i.e., the intimal area, the medial area and the ratio of the maximum:minimum wall thickness. Using these measurements, and intimal:medial ratio (i.e., I/M ratio) was also calculated.

### Example 7

### In Vitro Expression of PTEN From Grafted AdPTEN Saphenous Veins

This example verifies that AdPTEN saphenous vein graft tissue has stably transfected the PTEN gene by in vitro expression.

Prior to the fixation step according to Example 6, tissue samples were incubated in lysis buffer with 100 rpm shaking at 55° C. overnight (100 mM Tris:HCl, 5 mM EDTA, 0.2% SDS, 200 mM NaCl, 0.2 mg/ml Proteinase K (Sigma, Saint Louis Mo.). The supernatant was extracted with phenol:chloroform:isoamyl alcohol (25:24:1)(Sigma, Saint Louis Mo.) and the DNA was precipitated and diluted to 0.1 ìg/ìl. The polymerase chain reaction mixture consisted of: 1×Taq reaction buffer, 1.5 mM MgCl₂, 0.2 mM dNTPs (Roche), 1 ng/ìl primer, 2.5 units Taq polymerase (Invitrogen Life Technologies, Carlsbad Calif.) and 0.1-0.3 ìg DNA. A forward primer specific for the cytomegalovirus promoter and a reverse primer specific for human PTEN were utilized to amplify the transgene. Reaction conditions were: i) 95° C.×5 minutes; ii) 95° C.×30 seconds 6 59° C.×30 seconds 6 72° C.×1 minute (25-30 cycles); and iii) 72° C.×7 minutes (BioRad MyCycler; Biorad, Hercules Calif.).

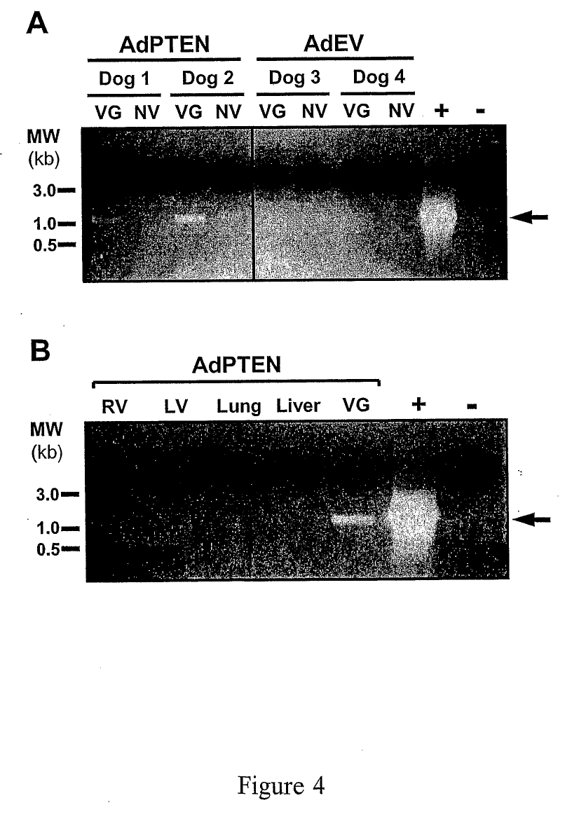

Total RNA isolated from samples of saphenous vein grafts, normal saphenous veins, left and right ventricular myocardium, liver and lung samples from four AdPTEN-treated canines were reverse transcribed by the above PCR protocol. See FIG. 4. The PTEN transgene cDNA was detected in all saphenous vein samples but not in normal saphenous veins from the same animals (i.e., serving as a negative control). Tissue harvested from the AdEV-transfected saphenous vein grafts, left and right ventricular myocardium, liver and lung also showed an absence of PTEN transgene cDNA.

### Example 8

### AdPTEN Vascular Smooth Muscle Cell Cultures

This example provides data showing the transfection of the PTEN enzyme into tissue cultures of vascular smooth muscle cells.

Vascular smooth muscle cells were isolated from canine and human saphenous veins (approved by the Institutional Review Board Of Duke University) under sterile conditions. Briefly, the adventitia was stripped away and the intima removed by blunt dissection. The media was cut into 1 cm sections and placed in culture dishes containing a small amount of growth medium as previously described. Gao et al., “Surface Hydrolysis Of Poly(glycolic acid) Meshes Increases The Seeding Density Of Vascular Smooth Muscle Cells” J Biomed Mater Res 42:417-424 (1998). After ten days, veins were removed and monolayers of vascular smooth muscle cells trypsanized and passaged between 3-5 times.

The resultant preparation of vascular smooth muscle cells were grown in 12-well plates in DMEM/F12 HAM containing 10% fetal bovine serum. When the cells were nearly confluent, the medium was changed to 2% fetal bovine serum and virus vectors were added at a multiplicity of infection of 100. After 24 hours, the medium was changed to serum-free, followed by different treatments or stimuli as indicated. As a control in all experiments, an identical group of cells were not transfected with the virus vector, but incubated under identical conditions.

Following an overnight transfection of adenovirus, vascular smooth muscle cells were serum-starved for five hours and then simulated for five minutes with platelet derived growth factor (20 ng/ml; R&D Systems, Minneapolis Minn.). Cells were then lysed in Triton° lysis buffer and samples separated by SDS 8-16% polyacrylamide gel electrophoresis, and thereafter transferred to a nitrocellulose membrane. The membranes were Western blotted with the following antibodies: i) anti-PTEN monoclonal (clone A2B1, Santa Cruz Biotechnology, Santa Cruz Calif.); ii) anti-phospho-Akt (Ser⁴⁷³); iii) anti-Akt; iv) anti-phospho-p44/42 ERK (Thr²⁰²/Tyr²⁰⁴) & anti-p44/42 ERK (Cell Signaling Technologies, Beverly Mass.); and v) rat monoclonal anti-á-tubulin (clone YL1/2, Abcam, Cambridge Mass.).

To evaluate the effect of PTEN overexpression on platelet derived growth factor mediated DNA synthesis in vascular smooth muscle cell cultures, [³H]-thymidine incorporation was assayed as previously described. Huang et al., “PTEN Modulates Vascular Endothelial Growth Factor Mediated Signaling And Angiogenic Effects” J Biol Chem 277:10760-10766 (2002). Briefly, vascular smooth muscle cells were plated in triplicate in 12-well plates at a concentration of 20,000 cells/well. The following day, half the cells were transfected with ADPTEN or AdGFP and the other half were subjected to sham-transfection procedures. The next day, cells were quiesced in serum-free medium for another 24 hours. The medium was replaced with fresh serum-free medium with, or without, 20 ng/ml platelet-derived growth factor or 5% fetal bovine serum, and incubated for eighteen hours. The cells were then pulse-labeled with 2 ìCi/ml [³H]-thymidine (Amersham Biosciences, Piscataway, N.J.) for three hours and thymidine incorporation was assessed by liquid scintillation counting.

To determine cell count, unlabeled vascular smooth muscle cells were plated in triplicate on 12-well plates and half the cells were transfected with AdPTEN or AdGFP and the other half were subjected to sham-transfection procedures. The cells were then incubated in a serum-free medium for forty-eight hours either with, or without, 20 ng/ml platelet derived growth factor or 5% FBS. The cells were then trypsinized and counted on a hemacytometer (Fisher Scientific, La Jolla Calif.).

### Example 9

### PTEN Mechanism of Action in Vascular Smooth Muscle Cell Culture

This example presents data suggesting that the effects of PTEN on vascular smooth muscle cell growth are mediated in part by inhibition of protein kinase B (Akt), but not extracellular signal-regulated kinase (ERK).

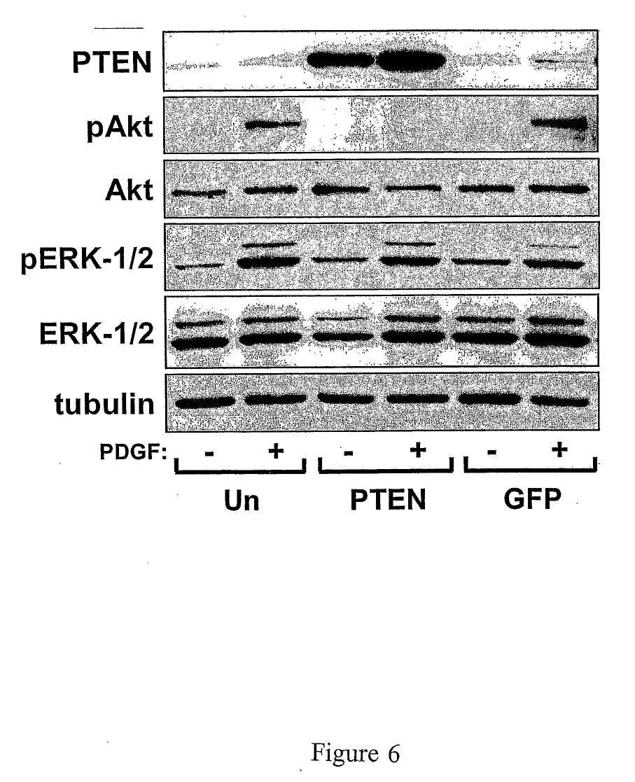

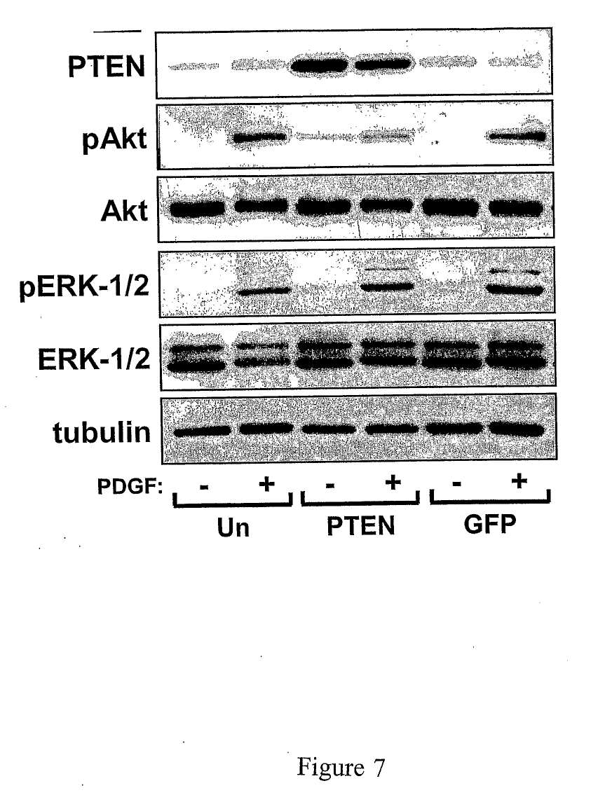

Human and canine vascular smooth muscle cells were cultured according to Example 8. The data demonstrate PTEN expression in control tissues and PTEN overexpression in AdPTEN-transfected cells. See FIG. 6 and FIG. 7. This PTEN overexpression was then evaluated for effects on known signaling pathways controlling vascular smooth muscle cell proliferation. For example, activation of Akt by PIK initiates a potent survival signaling cascade and platelet derived growth factor is known to stimulate this pathway. The data in FIGS. 6 & 7 show canine and human vascular smooth muscle cells responding to platelet derived growth factor by phosphorylation of Akt and ERK. During the overexpression of PTEN, however, the platelet derived growth factor induced Akt phosphorylation was inhibited but not the phosphorylation of ERK.

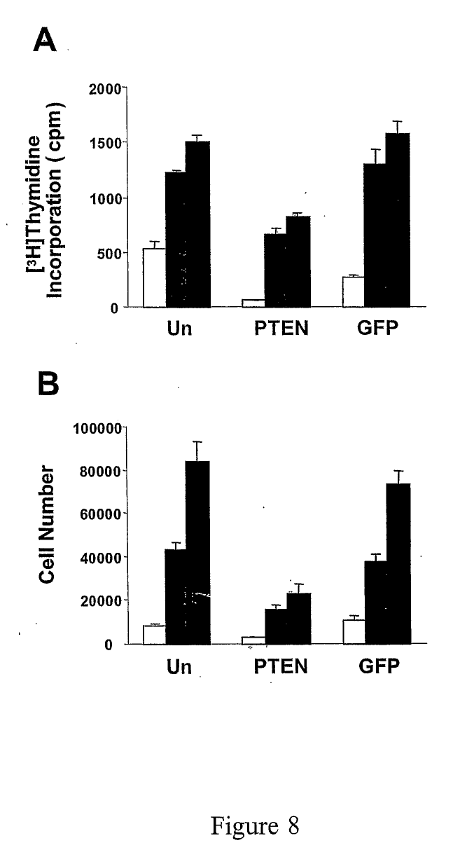

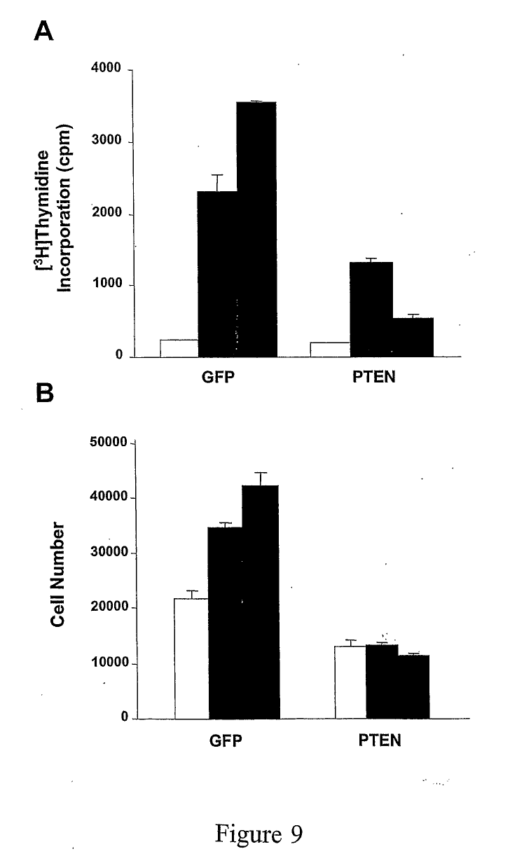

This experiment demonstrates that the effects of PTEN overexpression on PIK mediated signaling pathways are correlated with the cellular responses relevant to the process of intimal hyperplasia. [³H]-thymidine uptake was measured according to Example 8 in the presence of either platelet derived growth factor or fetal bovine serum (both are known to stimulate DNA synthesis). Platelet derived growth factor induced significant increases in DNA synthesis in sham-transfected controls and AdGFP-transfected canine vascular smooth muscle cell cultures (FIG. 8A). PTEN overexpression significantly decreased basal thymidine incorporation, as well as thymidine incorporation in response to platelet derived growth factor or fetal bovine serum stimulation. Similar data is presented regarding analogous experiments in human vascular smooth muscle cell cultures. See FIG. 9A.

Cell proliferation in response to either platelet derived growth factor or fetal bovine serum was confirmed by cell count procedures. See FIG. 8B. PTEN overexpression significantly decreased the number of unstimulated cells compared with AdGFP-transfected or sham-transfected cells suggesting a pro-apoptotic role for PTEN. Interestingly, in the human vascular smooth muscle cells, the number of AdPTEN-transfected cells in the platelet derived growth factor and the fetal bovine serum stimulated groups were still significantly less than the sham-transfected cells not treated with these DNA synthesis stimulating agents. Together, these findings demonstrate that PTEN overexpression blocks platelet derived growth factor and fetal bovine serum mediated increases in vascular smooth muscle cell proliferation; likely, in part, by promoting apoptosis.

### Example 10

### In Vivo AdPTEN Transfection into the External Carotid Artery

This example demonstrates the effectiveness of AdPTEN transfection during in vivo administration.

A rat carotid injury model and local adenovirus delivery was performed on 46 male Sprague-Dawley rats (450-500 gms). Lee et al., “In Vivo Adenoviral Vector-Mediated Gene Transfer Into Balloon-Injured Rat Carotid Arteries” Circ Res 73:797-807 (1993); Clowes et al., “Mechanisms Of Stenosis After Arterial Injury” Lab Invest 49:208-215 (1983). Following anesthetization (ketamine, 150 mg/kg) the right external and common carotid arteries were surgically exposed and isolated, and the endothelium of the common carotid artery was denuded with a 2Fr Fogart balloon catheter (Baxter Healthcare, Irving Calif.). After balloon removal, the common carotid artery was flushed with phosphate buffered saline and a 1-cm segment was isolated with vascular clamps. Adenovirus vector transfection was studied using 100 ìl injections into the common carotid artery of: i) PBS—sham-transfection; ii) AdPTEN (5×10⁹ pfu in PBS) and iii) AdEV (5×10⁹ pfu in PBS). After a thirty minute incubation time, each solution was removed and the external carotid artery was ligated and blood flow to the common carotid artery was restored.

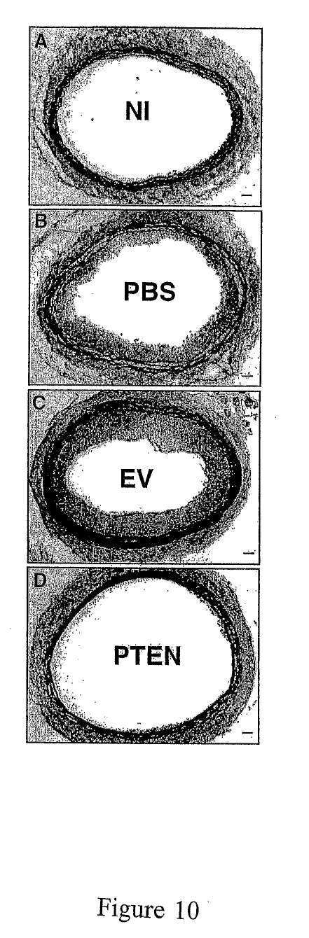

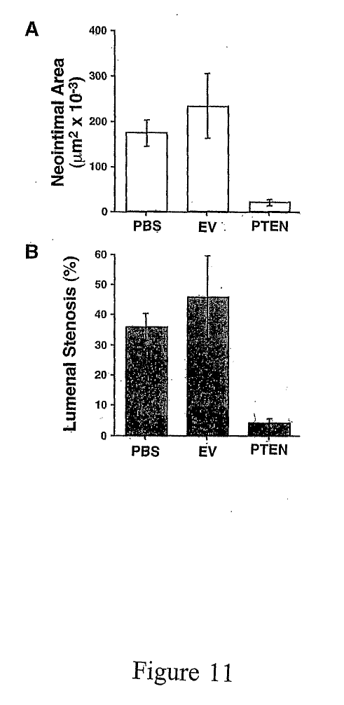

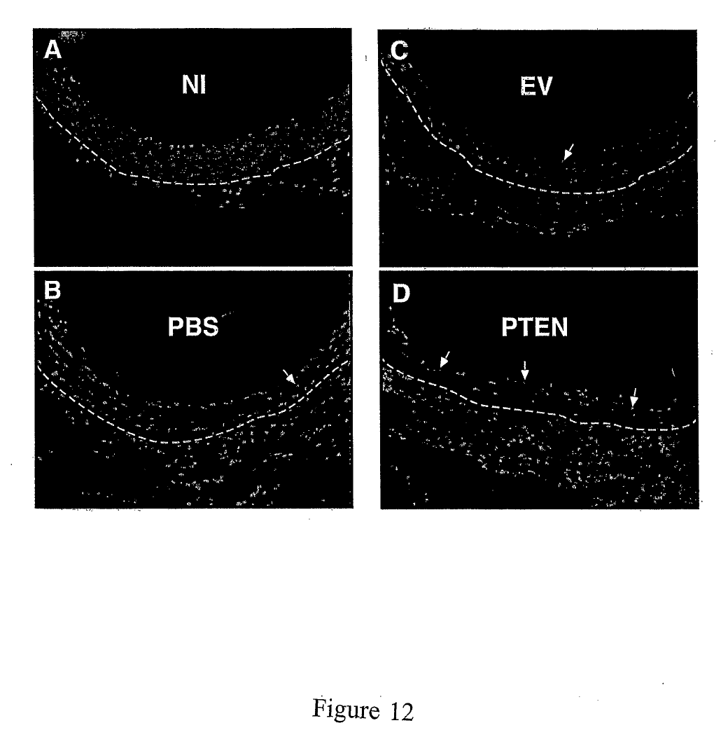

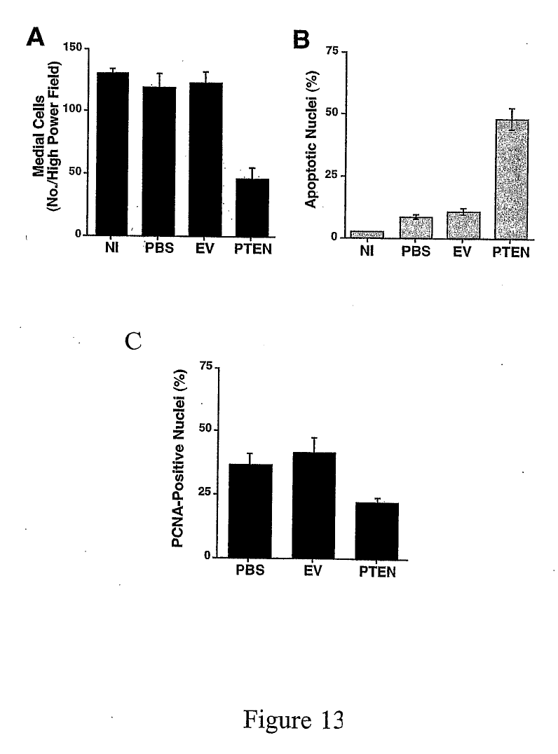

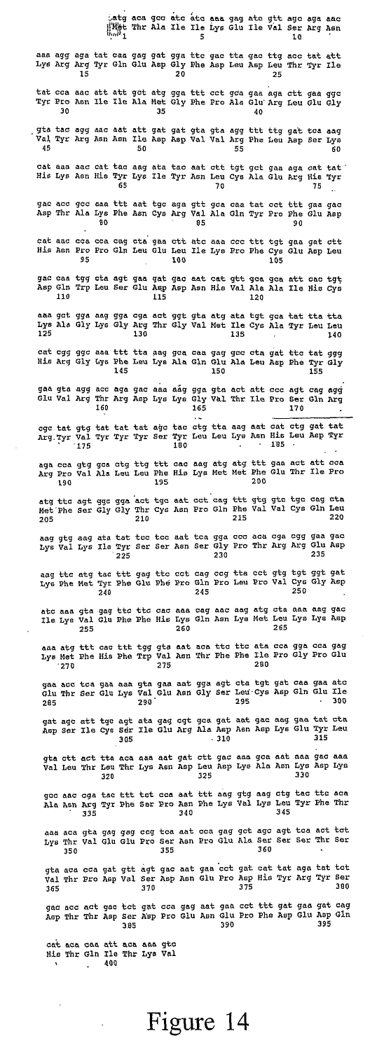

Fourteen days after the procedure, ADPTEN treatment reduced the neointimal area and stenosis. See FIGS. 10 & 11, respectively. The mean percent vessel stenosis in the AdPTEN-treated vessels was only 4″2% as compared to 36″4% for sham-transfected and 46″14% for AdEV-transfected vessels. The morphological data show changes in nuclear morphology and expression of proliferating cell nuclear antigen that are consistent with apoptosis. See FIGS. 12 & 13. Consistent with the slight medial thinning, AdPTEN-transfected vessels had an approximate 60% reduction in the total number of nuclei (FIGS. 12 and 13A), and almost 50% of these nuclei were fragmented or condensed (FIG. 13B). Moreover, AdPTEN treatment significantly reduced medial smooth muscle proliferation, as determined by proliferating cell nuclear antigen staining. See FIG. 13C.

We claim:

1. A method, comprising:
a) providing;
i) a patient comprising a saphenous vein and first and second arteries, said saphenous vein comprising smooth muscle cells;
ii) an adenoviral vector comprising a nucleic acid encoding a PTEN amino acid sequence, said amino acid sequence selected from the group consisting of SEQ ID NO:1 and derivatives of SEQ ID NO:1, said derivatives comprising amino acid sequences comprising substitutions, said substitutions selected from the group consisting of Ser 6 Glu, Thr 6 Glu, Asp 6 Asn and Cys 6 Ser;
b) removing at least a portion of said saphenous vein from said patient to create a removed vein portion, wherein said removed vein portion comprises a first end and a second end; and
c) contacting said removed vein portion ex vivo with said vector under conditions such that said PTEN sequence is introduced into a least a portion of said smooth muscle cells to create a treated vein portion.

2. The method of claim 1, wherein said amino acid sequence is proteasome resistant.

3. The method of claim 1, wherein said patient has cardiovascular disease.

4. The method of claim 3, wherein said cardiovascular disease comprises coronary artery disease.

5. The method of claim 1, further comprising (d) introducing said treated vein portion into said patient.

6. The method of claim 5, wherein said introducing comprises attaching said first end of said treated vein portion to said first artery under conditions such that a distal anastomosis is created.

7. The method of claim 5, wherein said introducing further comprises attaching said second end of said treated vein portion to said second artery under condition such that a proximal anastomosis is created.

8. The method of claim 5, wherein said first artery comprises a coronary artery.

9. The method of claim 6, wherein said second artery comprises the aorta.

10. A method, comprising:
a) providing;
i) a patient comprising a saphenous vein, a peripheral artery and a peripheral vein, said peripheral vein comprising smooth muscle cells;
ii) an adenoviral vector comprising a nucleic acid encoding a PTEN amino acid sequence, said amino acid sequence selected from the group consisting of SEQ ID NO:1 and derivatives of SEQ ID NO:1, said derivatives comprising amino acid sequences comprising substitutions, said substitutions selected from the group consisting of Ser 6 Glu, Thr 6 Glu, Asp 6 Asn and Cys 6 Ser;
b) removing at least a portion of said saphenous vein from said patient to create a removed vein portion, wherein said removed vein portion comprises a first end and a second end; and
c) contacting said removed vein portion ex vivo with said vector under conditions such that said PTEN sequence is introduced into a least a portion of said smooth muscle cells to create a treated vein portion.

11. The method of claim 10, wherein said patient requires hemodialysis.

12. The method of claim 10, wherein said hemodialysis comprises long-term maintenance.

13. The method of claim 9, further comprising (d) introducing said treated vein portion into said patient to create an arterio-venous graft.

14. The method of claim 13, wherein said introducing comprises attaching said first end of said treated saphenous vein portion to said peripheral artery under conditions such that a first anastomosis is created.

15. The method of claim 13, wherein said introducing further comprises attaching said second end of said treated vein portion to said peripheral vein under conditions such that a second anastomosis is created.

16. The method of claim 13, wherein said arterio-venous graft is selected from the group consisting of a wrist radiocephalic graft, a forearm radiocephalic graft and an antecubital brachiocephalic graft.

17. A method, comprising:
a) providing;
i) a patient comprising a peripheral artery and a peripheral vein, said peripheral vein comprising smooth muscle cells;
ii) an adenoviral vector comprising a nucleic acid encoding a PTEN amino acid sequence, said amino acid sequence selected from the group consisting of SEQ ID NO:1 and derivatives of SEQ ID NO:1, said derivatives comprising amino acid sequences comprising substitutions, said substitutions selected from the group consisting of Ser 6 Glu, Thr 6 Glu, Asp 6 Asn and Cys 6 Ser;
b) connecting said peripheral artery and said peripheral vein such that an arterio-venous fistula is created:
c) contacting said arterio-venous fistula in vivo with said vector under conditions such that said PTEN sequence is introduced into a least a portion of said smooth muscle cells to create a treated arterio-venous fistula.

18. The method of claim 16, wherein said patient has a renal disease.

19. The method of claim 17, wherein said patient requires hemodialysis.

20. The method of claim 19, wherein said hemodialysis comprises long-term maintenance.

21. The method of claim 17, further comprising prior to step (c) ligating said arterio-venous fistula.

22. The method of claim 17, wherein said arterio-venous fistula is selected from the group consisting of a wrist radiocephalic fistula, a forearm radiocephalic fistula and an antecubitat brachiocephalic fistula.

23. A composition comprising an isolated tissue portion, said tissue portion being transfected by exposure to an adenovirus.

24. The composition of claim 23, wherein said adenovirus encodes at least a portion of a PTEN gene.

25. The composition of claim 23, wherein said tissue portion comprises a vascular tissue.
